(19) United States
(12) Patent Application Publication
(10) Pub. No.: US 2025/0381060 A1
(43) Pub. Date: Dec. 18, 2025

(54) ADJUSTABLE ELEVATING CHIN LIFT DEVICE AND METHODS OF USE

(71) Applicant: Venkata Buddharaju, Park Ridge, IL (US)

(72) Inventor: Venkata Buddharaju, Park Ridge, IL (US)

(21) Appl. No.: 18/744,090

(22) Filed: Jun. 14, 2024

(51) Int. Cl. A61F 5/56

(52) U.S. Cl. CPC A61F 5/56

(57) ABSTRACT

The chin lift device includes a headgear portion that surrounds the top of the user's head and a bendable element that is positioned below and spaced from the tip of the chin during use. The headgear portion includes first and second connection points that are positioned in front of the user's ears when worn. First and second adjustment members are attached to the first and second connection points, respectively, and enable the user to adjust the tightness of the bendable element below the chin.

### BACKGROUND OF THE INVENTION

The present subject matter relates generally to a chin lift device that minimizes snoring. More specifically, the present invention relates to a chin lift device that lifts the mandible during use, which opens the user's airway and allows better airflow.

Conventional anti-snoring chin lift devices typically wrap around the head from the back of the parietal bone down to the tip of the chin. A chin strap portion covers portions of the front of the chin as well as the underside thereof. During use, the upward pulling force of the head gear is at the tip of the chin, which pushes the user's chin inwards toward the throat. Such positioning potentially compromises airflow on the back of the throat, reducing the posterior pharyngeal space and nasal airflow going into the lungs while sleeping.

Accordingly, there is a need for an anti-snoring chin lift device that allows better airflow in the back of the throat while minimizing snoring, as described herein.

### BRIEF SUMMARY OF THE INVENTION

To meet the needs described above and others, the present disclosure provides an anti-snoring chin lift device that allows better airflow in the back of the throat while minimizing snoring. The presently disclosed chin lift device includes a bendable element that extends below the user's chin approximately one inch behind the tip of the chin and is pulled upwardly during use.

By providing a bendable element approximately one inch behind the tip of the chin and pulls upwardly during use, the chin lift device reduces snoring while minimizing any impact on airflow through the back of the throat. The upward pulling force by the head gear is on the mandible below and behind the chin which rotates the temporomandibular joint (TMJ) upwards thereby pulling the jaw and chin forward to open the airway and allow better airflow in the back of the throat, thereby reducing snoring and potentially apneas during sleep. The chin lift device described herein may minimize snoring and/or sleep apnea, open the airway for post operative patients, improve a compromised airway for drowsy patients getting insufficient sleep particularly in the post operative recovery room, and reduce air leaks for users using continuous positive airway pressure (CPAP) or bilevel positive airway pressure (BIPAP) devices.

In an example embodiment, the chin lift device includes a headgear portion that surrounds the top of the user's head during use. The headgear portion includes first and second connection points that are positioned in front of the user's ears when worn. In some embodiments, first and second adjustment members are attached to the first and second connection points, respectively, and connect to the bendable element that extends between the first and second adjustment members and under the user's chin during use. In other embodiments, the bendable element attaches to the headgear portion.

In still further embodiments, the bendable element may be incorporated into a chin strap that covers a portion of the front of the chin as well as the underside of the chin. In this embodiment, the bendable element is positioned on the underside of the chin and is pulled upwards as described herein.

An object of the invention is to provide a device that minimizes snoring without adversely affecting airflow through the throat.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is a perspective view of a first embodiment of a chin lifting device of the present application.

FIG. 2 is a perspective view of the chin lifting device of FIG. 1 during use.

FIG. 3 is a perspective view of a second embodiment of the chin lifting device of the present application.

### DETAILED DESCRIPTION OF THE INVENTION

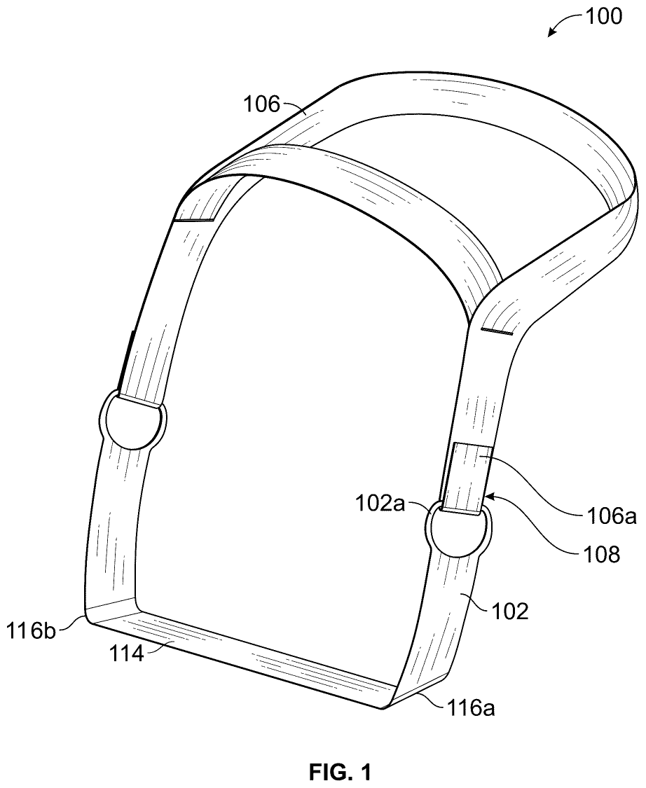

FIGS. 1-3 illustrate example chin lift devices 100, 200, each including a bendable element 102, 202 positioned below and behind the user's chin approximately one inch behind the tip of the chin 104, 2024 that is pulled upwardly.

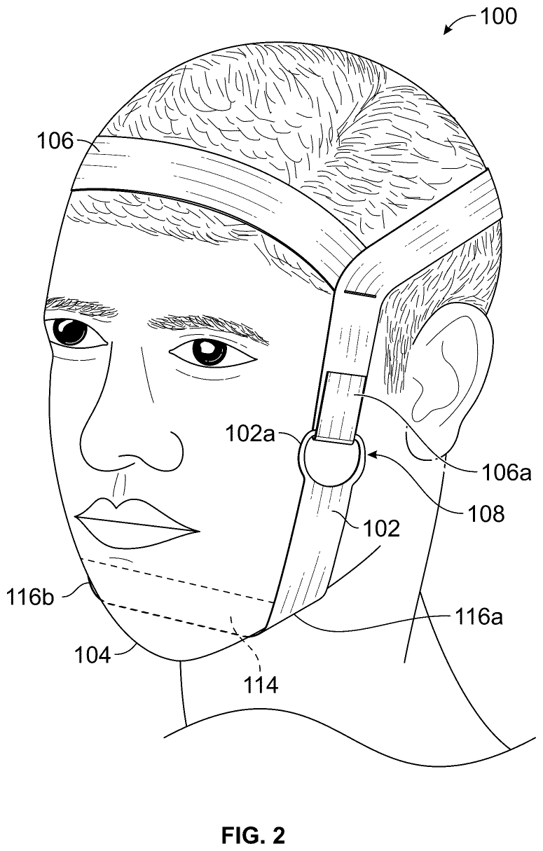

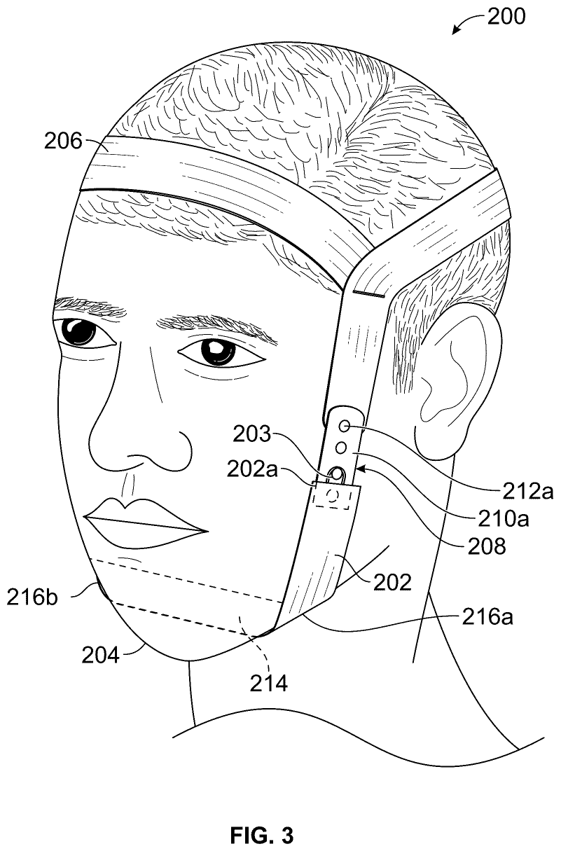

In both embodiments, the bendable element 102, 202 comprises a material is sufficiently rigid to provide resistance against the user's attempts to open the mouth while being sufficiently soft and bendable to conform to the shape of the user's jaw, particularly at the contact points 116, 216 (see FIGS. 2 and 3) described below. Generally, the bendable element 102, 202 exerts pressure on the underside of the mandible behind the chin and is stabilized during the pull. In some examples, the bendable element 102, 202 may comprise a soft metal, such as aluminum, or other suitably soft rigid material that maintains a shape or positioning. In other embodiments, the bendable element 102, 202 may be an aluminum or other soft metal material wrapped in silicone or other suitable housing material that is gentle on the skin.

The bendable element 102, 202 is sufficiently rigid to maintain a shape in order to exert pressure on the underside of the mandible. The bendable element is also sufficiently bendable to adapt to the mandible or head of the user. More specifically, the bendable element is sufficiently malleable to adapt to different mandible sizes, shapes, lengths and is able to curve around the mandible contact points 116, 216, so that it is relatively fixed when pulled by the head gear. In contrast, a different material such as cloth would distribute the force evenly such that pressure is not applied at a specific point or section on the mandible and cannot be malleable during use.

In both example embodiments, the chin lift device 100, 200 includes a headgear portion 106, 206 that surrounds the top of the user's head during use. For example, the headgear portion 106, 206 could include a first strap that surrounds the user's head along the forehead and a second strap that crosses the top or crown of the user's head.

The headgear portion 106, 206 includes first and second connection points 108, 208 that are adjacent to the user's ears and jaw when worn. In some embodiments, these connection points 108, 208 are at the mandible and below the temporal bone. The connection points 108, 208 are positioned ideally below the ears above the jawbone as shown in FIGS. 2 and 3, such that the force pulling the bendable element 102, 202 causes the mandible to be pulled upwards. In one example embodiment, the connection point 108, 208 is positioned about an inch below a bottom of the ear along the ramus of the mandible.

In the first embodiment, each end of the bendable element 102 includes a ring structure 102a through which an end 106a of the headgear portion 106 connects at the connection point 108. The end 106a extends through the ring structure 102a of the bendable element 102 and attaches to itself as shown in FIGS. 1 and 2. In the illustrated embodiment, the end 106a of the headgear portion 106 includes hook and loop material such as Velcro®, which allows the user to adjust the tightness and positioning of the bendable element 102 during use.

As an alternative to the ring structure, the bendable element 102 may attach to the headgear portion 106 in any other suitable means that allow for the user to adjust the tightness of the bendable element 102 on the underside of the chin during use in still further embodiments. For example, the ends of the bendable element 102 may include a loop, a hook, a button, hook and loop material (such as Velcro®), or the like that engages with the ends 106s of the headgear portion 106.

In the second embodiment, the headgear 206 includes first and second adjustment members 210a, 210b at the first and second connection points 208a, 208b, respectively. The illustrated embodiment shows only the first adjustment member 210a at the first connection point 208a. Each adjustment member 210 attaches to the headgear portion 206 by any securement means such as a clip, a button, hook and loop material (Velcro®), being sewn into place, or the like.

In the embodiment illustrated in FIG. 3, the adjustment member 210a includes a plurality of adjustment points or protrusions 212a that allow the user to adjust the positioning and tightness of the bendable element 202 against the user's chin 204. The ends 202a, 202b of the bendable element 202 connect to the selected adjustment point 112 of the adjustment member 110 through the use of a loop, a hook, a button, hook and loop material (such as Velcro®), or the like. In FIG. 3, the end 202a of the bendable element 202 includes a loop 203 comprising an elastic band that wraps around or capture a protrusion 212 on the adjustment member 210 to maintain a taut positioning of the bendable element 202 below the user's chin 204. In still further embodiments, the adjustment member 210a includes a plurality of adjustment openings, and each end 202a, 202b of the bendable element 202 includes a protrusion that snap-fits into an adjustment opening of the plurality of adjustment openings. In other embodiments, alternative adjustment mechanisms may be used.

In both illustrated embodiments of the chin lift device 100, 200, the bendable element 102, 202 is a body of material having a generally concave shape between first and second opposing ends 102, 202. In some embodiments, the body includes a chin support bend 114, 214 between first and second contact points 116a, 116b, 216a, 216b located between the first and second ends of the body. In the illustrated embodiments, the contact points 116, 216 at which the bendable element 102, 202 contacts the user's jawbone can be adjusted based on the shape and size of the user's mandible as described below.

Specifically, the chin support bend 114, 214 is a portion of the body of the bendable element that extends between the first and second contact points 116a, 116b, 216a, 216b during use. As shown in the illustrated embodiment of FIG. 1, the chin support bend 114, 214 is generally planar between the contact points 116, 216. In other embodiments, the chin support bend 114, 214 has a concave or other shape as desired. During use, the chin support bend 114, 214 is positioned approximately one inch behind the chin tip.

Also during use, the first and second contact points 116a, 116b, 216a, 216b of the chin support bend 114, 214 are generally positioned on the sides of the mandible bone as shown in FIGS. 2 and 3. The user can adjust the positioning of the contact points 116, 216 of the bendable element 102, 202 to wrap around the user's mandible, as shown in FIGS. 2 and 3. the user adjusts the contact points 116, 216 at the mandible bone to different sizes, shapes and lengths of the mandible to snugly fit the bendable element 102, 202 to the user's jaw.

In the illustrated embodiment, the bendable element 102, 202 is generally concave between the first and second ends 102a, 102b, 202a, 202b. The planar aspect of the chin lift portion 114, 214 conforms to the shape of the user's mandible as part of the generally concave shape. In such embodiments, the contact points 116, 216 may be adjusted and the body of the bendable element 102, 202 may be modified as desired.

In some example embodiments, the bendable element 102, 202 has a width spanning side edges of the body of about 0.5 in. to 1 in., although other shapes and sizes may be used depending on the size of the user and as desired. During use, a first edge of the bendable element is approximately 1 in. from the tip of the user's chin.

During use, the headgear portion 106, 206 is soft and comfortable while wearing during sleep. Care is taken to avoid positioning the headgear in the forehead area to prevent the headgear portion from slipping onto the face.

It should be noted that various changes and modifications to the presently preferred embodiments described herein will be apparent to those skilled in the art. Such changes and modifications may be made without departing from the spirit and scope of the present invention and without diminishing its attendant advantages.

We claim:

1. A chin lift device configured to be worn on a head of a user, the chin lift device comprising:
a headgear portion configured to mount to a top of the head, wherein the headgear portion includes first and second ends configured to be positioned near ears of the head; and
a bendable element including a body extending between a first end and a second end, wherein the first end is configured to connect to the first end of the headgear portion, and wherein the second end is configured to connect to the second end of the headgear portion;
wherein the bendable element is sufficiently rigid to maintain a shape, and wherein the bendable element is sufficiently bendable to adapt to the head of the user.

2. The chin lift device of claim 1, wherein the first end of the bendable element includes a first ring structure that connects to the first end of the headgear portion, and wherein the second end of the bendable element includes a second ring structure that connects to the second end of the headgear portion.

3. The chin lift device of claim 2, wherein the first and second ends of the headgear portion include attachment members configured to engage with the first ring structure and the second ring structure, respectively.

4. The chin lift device of claim 3, wherein each of the first and second ends of the headgear portion includes hook and loop material such that each end extends through and around the respective ring structure.

5. The chin lift device of claim 1, wherein the bendable element has a generally concave shape between the first and second ends, and wherein the bendable element includes a chin support bend having a generally planar shape.

6. A chin lift device configured to be worn on a head of a user, the chin lift device comprising:
a headgear portion configured to mount to a top of the head, wherein the headgear portion includes first and second connection points configured to be positioned near ears of the head;
a first adjustment member and a second adjustment member connected to the first connection point and the second connection point, respectively, of the headgear portion, wherein each of the first adjustment member and the second adjustment member include a plurality of first adjustment points and a plurality of second adjustment points, respectively; and
a bendable element including a body extending between a first end and a second end, wherein the first end connects to a first adjustment point of the first adjustment member, and wherein the second end connects to a second adjustment point of the second adjustment member.

7. The chin lift device of claim 6, further comprising connecting elements for connecting the first end and the second end to the first adjustment point and the second adjustment point.

8. The chin lift device of claim 7, wherein the connecting elements are selected from the group consisting of loops, hooks, buttons, and Velcro.

9. The chin lift device of claim 8, wherein the bendable element is configured to be positioned on an underside of the user's chin.

10. The chin lift device of claim 9, wherein the bendable element is configured to be positioned on the underside approximately one inch from a tip of the user's chin.

11. The chin lift device of claim 10, wherein the bendable element is configured to lift a jaw of the user upwards during use.

12. The chin lift device of claim 11, wherein the bendable element is configured to minimize forces applying the chin backwards toward a throat of the user.
